(19) United States
(12) Patent Application Publication
(10) Pub. No.: US 2024/0050631 A1
(43) Pub. Date: Feb. 15, 2024

(54) ANTI-MICROBIAL MEDICAL GRADE POLYMER SUBSTRATES WITH ANTI-FUNGAL AND ANTI-BACTERIAL PROPERTIES

(71) Applicant: University of Georgia Research Foundation, Inc., Athens, GA (US)

(72) Inventors: Hitesh HANDA, ATHENS, GA (US); Ryan P. DEVINE, Athens, GA (US); Megan Elizabeth BROOKS, Athens, GA (US)

(21) Appl. No.: 18/016,095

(22) PCT Filed: Jul. 14, 2021
(86) PCT No.: PCT/US2021/041610

§ 371 Date: Jan. 13, 2023

Related U.S. Application Data
(60) Provisional application No. 63/052,668, filed on Jul. 16, 2020.

(51) Int. Cl. A61L 29/08; A61L 29/12; A61L 29/16

(52) U.S. Cl. CPC A61L 29/085; A61L 29/126; A61L 29/16; A61L 2420/06; A61L 2300/114; A61L 2300/404; A61L 2300/216

(57) ABSTRACT

Described herein are substrates, devices, methods for treating and preventing bacterial and fungal infections, such as infections associated with medical devices, and the like. Materials, substrates and devices of the present disclosure include materials, such as medical grade polymers, having a coating/layer of polyene antimycotic molecules coupled to a surface of the polymer substrate. In certain aspects, substrates and devices of the present disclosure also include a nitric oxide (NO) releasing material also embedded in/coated on the material. Methods of the present disclosure includes methods of making the compositions and/or devices of the present disclosure including materials functionalized with polyene antimycotic molecules and NO releasing materials. Embodiments also include methods of using the materials, substrates, and devices of the present disclosure to treat/prevent fungal and/or bacterial infections in a subject, particularly infections associated with the use of a medical device.

### CROSS-REFERENCE TO RELATED APPLICATIONS

This application claims the benefit of and priority to co-pending U.S. Provisional Patent Application No. 63/052,668, filed on Jul. 16, 2020, the contents of which are incorporated by reference herein in their entireties.

### STATEMENT REGARDING FEDERALLY SPONSORED RESEARCH OR DEVELOPMENT

This invention was made with Government support under Grant R01 HL134899 awarded by the National Institutes of Health. The Government has certain rights in the invention.

### BACKGROUND

Approximately 50% of nosocomial infections are related to medical device use.¹ Pathogens can readily colonize into biofilms on the surface of indwelling devices, forming highly structured, often polymicrobial networks of microorganisms embedded within a protective three-dimensional extracellular matrix. Infection is largely managed through the administration of antibiotics, but the emergence of antibiotic resistance has made treatment increasingly difficult. One mechanism pathogens use to increase resistance is the formation of biofilms.² Biofilm matrices have been reported to increase drug resistance by 1000-fold compared to planktonic counterparts, and 60-70% of hospital-acquired infections involve biofilm formation, resulting in an additional $11 billion in healthcare costs in the US alone.³⁻⁴ Biofilms on indwelling medical devices often disseminate, leading to widespread bloodstream infections. Systemic administration of antibiotic and antifungals are frequently used to treat these infections, but difficulty in penetrating biofilms has led to increased antimicrobial resistance, which ultimately can necessitate additional surgeries to remove the infected device.

Although significant effort has been placed in combating bacterial pathogens, substantially less has been focused on combating device-related fungal infections. Candida species are the most common opportunistic fungal pathogens globally, and are the fourth leading cause of bloodstream infections in the US with a reported mortality rate of up to 50%.⁵⁻⁷ An increasing number of device-related infections involve Candida species, accounting for $2 billion in healthcare costs yearly.⁸ Once formed, eradication of biofilms via therapeutic interventions is seldom successful, requiring device removal. The urgent need to reduce the risk of infection has led to the incorporation of various antibacterial agents such as nitric oxide (NO) donors and antibiotics into various medical-grade polymers, but have shown minimal success against fungal pathogens, showing ineffectiveness or requiring high concentrations of NO.⁹⁻¹¹

Intravenous infusion of antifungal agents such as amphotericin B (AmB) can control fungal infections associated with indwelling devices, but is normally only administered after symptoms don't resolve after 3-7 days of antibiotic therapy.¹² Moreover, AmB infusion can result in side effects such as nephrotoxicity and rigors, ultimately resulting in treatment withdrawal.¹²⁻¹³ Although amphotericin B is considered first line therapy for invasive mucormycosis infections, cryptococcal meningitis, and certain aspergillus and candidal infections, it is well known for its severe and potentially lethal side effects. Very often, it causes a serious reaction soon after infusion (within 1 to 3 hours), consisting of high fever, shaking chills, hypotension, anorexia, nausea, vomiting, headache, dyspnea and tachypnea, drowsiness, and generalized weakness. Thus, systemic treatment with AmB is typically reserved for only the most severe infections in critically ill or immunocompromised patients.

### SUMMARY

Described herein are substrates, devices, methods for treating and preventing bacterial and fungal infections, such as infections associated with medical devices, and the like. Materials, substrates and devices of the present disclosure include materials, such as medical grade polymers, having a coating/layer of polyene antimycotic molecules coupled to a surface of the polymer substrate. In certain aspects, substrates and devices of the present disclosure also include a nitric oxide (NO) releasing material also embedded in/coated on the material. Methods of the present disclosure includes methods of making the compositions and/or devices of the present disclosure including materials functionalized with polyene antimycotic molecules and NO releasing materials. Embodiments also include methods of using the materials, substrates, and devices of the present disclosure to treat/prevent fungal and/or bacterial infections in a subject, particularly infections associated with the use of a medical device.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic illustration of synthesis of AmB surface coating (with optional pre-step of SNAP impregnation for a SNAP-AmB treated polymer through solvent swelling (SNAP impregnation—step 1)) with EDC/NHS coupling and AmB immobilization (steps 2-4).

FIG. 2 is a bar graph illustrating average real-time NO release of SNAP and SNAP-AmB PDMS at 37° C. submerged in PBS (n=6). Data is reported in mean±standard deviation. Statistical significance (*) was calculated between SNAP and SNAP-AmB surfaces for each time point (p<0.05).

FIGS. 3A-3C is a series of bar graphs illustrating adhered bacterial and fungal viability after 24 h exposure of materials to S. aureus (A), E. coli (B), and C. albicans (C) quantified in CFU/cm². Statistical significance (p<0.05) is indicated by *, %, and # compared to PDMS, AmB, and SNAP samples, respectively. Measurements are reported in mean±SD.

FIG. 4. is a graph illustrating in vitro platelet adhesion normalized to surface area. Statistical significance (p<0.05) is indicated by *, %, and # compared to PDMS, AmB, and SNAP samples, respectively. Measurements are reported in mean±SD.

FIG. 5. illustrates in vitro cytotoxicity measurements against human fibroblasts. Statistical significance (p<0.05) is indicated by *, %, and # compared to PDMS, AmB, and SNAP samples, respectively. Measurements are reported mean±SD.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

In describing and claiming the disclosed subject matter, the following terminology will be used in accordance with the definitions set forth below.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an inert excipient” includes, but are not limited to, mixtures or combinations of two or more such inert excipients, and the like.

It should be noted that ratios, concentrations, amounts, rates, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed and “about 5 to about 15” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y” ’, where ‘x’ and ‘y’ are numerical values, includes “about ‘x’ to about ‘y” ’.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

It should be noted that ratios, concentrations, amounts, rates, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance and instances where it does not.

As used herein, the term “biocompatible,” with respect to a substance or fluid described herein, indicates that the substance or fluid does not adversely affect the short-term viability or long-term proliferation of a target biological particle within a particular time range.

The terms “antimicrobial” and “antimicrobial characteristic” refers to the ability to kill and/or inhibit the growth of microorganisms. A substance having an antimicrobial characteristic may be harmful to microorganisms (e.g., bacteria, fungi, protozoans, algae, and the like). A substance having an antimicrobial characteristic can kill the microorganism and/or prevent or substantially prevent the growth or reproduction of the microorganism.

The term “antimicrobial effective amount” as used herein refers to that amount of the compound being administered/released which will kill microorganisms or inhibit growth and/or reproduction thereof to some extent (e.g. from about 5% to about 100%). In reference to the compositions or articles of the disclosure, an antimicrobial effective amount refers to that amount which has the effect of diminishment of the presence of existing microorganisms, stabilization (e.g., not increasing) of the number of microorganisms present, preventing the presence of additional microorganisms, delaying or slowing of the reproduction of microorganisms, and combinations thereof. Similarly, the term “antibacterial effective amount” refers to that amount of a compound being administered/released that will kill bacterial organisms or inhibit growth and/or reproduction thereof to some extent (e.g., from about 5% to about 100%). In reference to the compositions or articles of the disclosure, an antibacterial effective amount refers to that amount which has the effect of diminishment of the presence of existing bacteria, stabilization (e.g., not increasing) of the number of bacteria present, preventing the presence of additional bacteria, delaying or slowing of the reproduction of bacteria, and combinations thereof.

The terms “bacteria” or “bacterium” include, but are not limited to, Gram positive and Gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chryseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydia trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Prevotella species, and Cowdria ruminantium, or any strain or variant thereof. The Gram-positive bacteria may include, but is not limited to, Gram positive Cocci (e.g., Streptococcus, Staphylococcus, and Enterococcus). The Gram-negative bacteria may include, but is not limited to, Gram negative rods (e.g., Bacteroidaceae, Enterobacteriaceae, Vibrionaceae, Pasteurellae and Pseudomonadaceae). In an embodiment, the bacteria can include Mycoplasma pneumoniae.

The terms “fungus” or “fungi” include, any fungal organisms susceptible to treatment with AmB, such as but not limited to the following: Candida, Aspergillus, Cryptococcus, Zygomycetes, Histoplasma, Fusarium, Sporothrix, Blastomyces dermatitidis, Leishmania, Cryptococcus, and Coccidioides immitis.

As used herein, the term “subject” includes humans, mammals (e.g., cats, dogs, horses, etc.), birds, and the like. Typical subjects to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a host. The term “living host” refers to the entire host or organism and not just a part excised (e.g., a liver or other organ) from the living host.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable. In addition, “treat”, “treating”, and “treatment” can also be therapeutic in terms of a partial or complete cure for a disease and/or adverse effect attributable to the disease. As used herein, the terms “prevent,” “prophylactically treat,” or “prophylactically treating” refers to completely, substantially, or partially preventing a disease/condition or one or more symptoms thereof in a host. Similarly, “delaying the onset of a condition” can also be included in “preventing/prophylactically treating”, and refers to the act of increasing the time before the actual onset of a condition in a patient that is predisposed to the condition.

As used herein, “endolumenally,” “intraluminally” or “transluminal” all refer synonymously to implantation placement by procedures wherein the prosthesis is advanced within and through the lumen of a body vessel from a remote location to a target site within the body vessel. In vascular procedures, a medical device will typically be introduced “endovascularly” using a catheter over a wire guide under fluoroscopic guidance. The catheters and wire guides may be introduced through conventional access sites to the vascular system.

Unless otherwise indicated, as used herein, a “layer” refers to a portion of a structure having a defined composition or structure and a defined boundary with respect to an adjacent material. A layer of a graft material may be deposited by spray deposition of a polymer solution in multiple spray deposition events. For example, a single layer may be formed by deposition of material in separate portions, where no definite boundary of structure or composition is present between the material deposited in the first and subsequent portions. Furthermore, a single layer may be formed by spray deposition of a first portion of a deposited material followed by drying of the deposited material and subsequent spray deposition of a second portion of material with the same composition onto the dried deposited material, provided that the deposited material does not include a structural or compositional boundary between the first deposited material and the second deposited material.

The term “luminal surface” or “luminal side,” as used herein, refers to the portion of the surface area of a medical device defining at least a portion of an interior lumen. Conversely, the term “abluminal surface” or “abluminal side,” as used herein, refers to portions of the surface area of a medical device that do not define at least a portion of an interior lumen. For example, where the medical device is a tubular frame formed from a plurality of interconnected struts and bends defining a cylindrical lumen, the abluminal surface can include the exterior surface, sides and edges of the struts and bends, while the luminal surface can include the interior surface of the struts and bends.

The term “substituted” refers to any one or more hydrogens on the designated atom that can be replaced with a selection from the indicated group, provided that the designated atom's normal valence is not exceeded.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic.

The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “substituted,” as in “substituted alkyl”, means that the substituted group may contain in place of one or more hydrogens a group such as alkyl, hydroxy, amino, halo, trifluoromethyl, cyano, —NH(alkyl), —N(alkyl)₂, alkoxy, alkylthio, or carboxy, and thus embraces the terms haloalkyl, alkoxy, fluorobenzyl, and the sulfur and phosphorous containing substitutions referred to below.

The terms “amine” and “amino” are art-recognized and refer to both unsubstituted and substituted amines, e.g., a moiety that can be represented by the general formula:

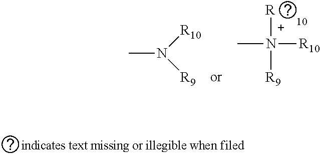

wherein R₉, R₁₀, and R′₁₀ each independently represent a hydrogen, an alkyl, an alkenyl, —(CH₂)ₘ—R₃ or R₉ and R₁₀ taken together with the N atom to which they are attached complete a heterocycle having from 4 to 8 atoms in the ring structure; R₃ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloalkyl, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “alkyl amino group” is an alkyl group as defined herein substituted with one or more amino groups.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. For example, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group.

The term “amide” as used herein is represented by the formula —NHC(O)A¹ or —C(O)NHA¹, where A¹ can be alkyl, cycloalkyl, alkenyl, cycloalkenyl, alkynyl, cycloalkynyl, aryl, or heteroaryl group.

A “polyene antimycotic molecule” sometimes referred to as polyene antibiotics, are a class of antimicrobial polyene compounds that target fungi. In certain aspects, the polyene antimycotic molecule has a carboxylic acid or ester group that can react with an amine to produce an amide. Examples of polyene antimycotic molecules include amphotericin B (AmB), nystatin, natamycin, or hamycin.

Abbreviations: NO, nitric oxide; SNAP, S-nitroso-N-acetylpenicillamine; NAP, N-acetyl-D-penicillamine; 3-Acetamido-4,4-dimethylthietan-2-one, NAP-thiolactone; LB, Luria broth; EDTA, ethylenediamine tetraacetic acid; CarboSil® 20 80A thermoplastic silicone-polycarbonate-urethane (hereafter will be referred to as CarboSil); THF, tetrahydrofuran; PBS, Phosphate buffered saline; ATCC, American Type Tissue Collection. GSNO, S-nitroso-glutathione; SIM, Surface immobilized; RSNO, S-nitrosothiol.

### DISCUSSION

In accordance with the purpose(s) of the present disclosure, as embodied and broadly described herein, embodiments of the present disclosure, in some aspects, relate to compositions/material, substrates, devices, methods for treating and preventing bacterial and fungal infections, such as infections associated with medical devices, and the like. Materials, substrates and devices of the present disclosure include materials, such as medical grade polymers, having a coating/layer of polyene antimycotic molecules coupled to a surface of the polymer substrate. In embodiments, substrates and devices of the present disclosure also include a nitric oxide (NO) releasing material also embedded in/coated on the material. Methods of the present disclosure includes methods of making the compositions and/or devices of the present disclosure including materials functionalized with polyene antimycotic molecules and NO releasing materials. Embodiments also include methods of using the materials, substrates, and devices of the present disclosure to treat/prevent fungal and/or bacterial infections in a subject, particularly infections associated with the use of a medical device.

As discussed above, nosocomial infections related to medical device use present a serious challenge to many medical treatment plans. Medical device-related infections largely constitute hospital acquired infections; for example, a study on nosocomial infections in the ICU found that 95% of all UTIs are catheter-associated UTIs, 87% of bloodstream infections are catheter-associated bloodstream infections, and 86% of pneumonia is related to mechanical ventilator use.⁵⁵ While resistant bacterial infections are being addressed by a variety of approaches, persistent and severe fungal infections (and/or combined bacterial and fungal infections) present another challenge. While polyene antimycotic molecules such as, for example, amphotericin B is an antifungal medication used for serious fungal infections and leishmaniasis, it has serious drawbacks that limit its use.

One of the main applications of amphotericin B is for treating a wide range of systemic fungal infections. However, due to its extensive side effects, it is often reserved for severe infections in critically ill, or immunocompromised patients. It is considered first line therapy for invasive mucormycosis infections, cryptococcal meningitis, and certain aspergillus and candidal infections. It has been a highly effective drug for over fifty years in large part because it has a low incidence of drug resistance in the pathogens it treats. This is because amphotericin B resistance requires sacrifices on the part of the pathogen that make it susceptible to the host environment, and too weak to cause infection.

Although its mechanism of action is not fully known, it is believed that amphotericin B binds with ergosterol, a component of fungal cell membranes, forming pores that cause rapid leakage of monovalent ions (K+, Na+, H+ and Cl−) and subsequent fungal cell death, which may be AmB's primary effect as an antifungal agent. Researchers have found evidence that amphotericin B also causes oxidative stress within the fungal cell, but it remains unclear to what extent this oxidative damage contributes to the drug's effectiveness. The addition of free radical scavengers or antioxidants can lead to amphotericin resistance in some species, such as Scedosporium prolificans, without affecting the cell wall.

The severe and potentially lethal side effects of AmB are well known. Very often, it causes a serious reaction soon after infusion (within 1 to 3 hours), including symptoms such as high fever, shaking chills, hypotension, anorexia, nausea, vomiting, headache, dyspnea and tachypnea, drowsiness, and generalized weakness. The violent chills and fevers have caused the drug to be nicknamed “shake and bake”. This reaction sometimes subsides with later applications of the drug and may in part be due to histamine liberation. This nearly universal febrile response necessitates a critical (and diagnostically difficult) professional determination as to whether the onset of high fever is a novel symptom of a fast-progressing disease, or merely the effect of the drug. To decrease the likelihood and severity of the symptoms, initial doses are typically low and increased slowly. Paracetamol, pethidine, diphenhydramine, and hydrocortisone have all been used to treat or prevent the syndrome, but the prophylactic use of these drugs is often limited by the patient's condition.

Intravenously administered amphotericin B in therapeutic doses has also been associated with multiple organ damage. Kidney damage is a frequently reported side effect and can be severe and sometimes irreversible. Less kidney toxicity has been reported with liposomal formulations (such as AmBisome), and these formulations have become preferred in patients with preexisting renal injury. The integrity of the liposome is disrupted when it binds to the fungal cell wall, but is not affected by the mammalian cell membrane, so the association with liposomes decreases the exposure of the kidneys to amphotericin B, leading to a less nephrotoxic effect. However, other less-toxic anti-fungal treatments are needed for treating and preventing a broader range of microbial infections. Also needed are applications of polyene antimycotic molecules such as AmB that do not, due to risk of severe side effects, have to be a treatment of last resort.

Nitric oxide-releasing (NORel) materials have been developed over the past 30 years after the discovery of NO as an important signaling molecule in a number of biological processes, of which include acting as a strong bactericidal and antithrombotic agent.¹⁶⁻¹⁸ To mimic the physiological release of NO from the endothelium, various NO donors (such as S-nitrosothiols¹⁸⁻²⁰ and diazeniumdiolates²¹⁻²³) have been developed and can be integrated into polymeric materials for localized delivery of NO. Multiple methods have been used to integrate S-nitrosothiols such as S-nitroso-N-acetylpenicillamine (SNAP) into various medical grade polymers, and include physical blending within the polymer¹⁸⁻²⁴, immobilization to the polymer backbone²⁵⁻²⁶, or swelling into the polymer matrix²⁷⁻²⁸.

Thus, the materials, devices, and methods of the present disclosure provide an effective and non-toxic application of polyene antimycotic molecules such as AmB for treatment and prevention of fungal as well as bacterial infections, such as those associated with medical devices. The materials, devices, and methods of the present disclosure also provide for a combined anti-fungal and anti-bacterial effect with the inclusion of both polyene antimycotic molecules and NO-releasing compounds.

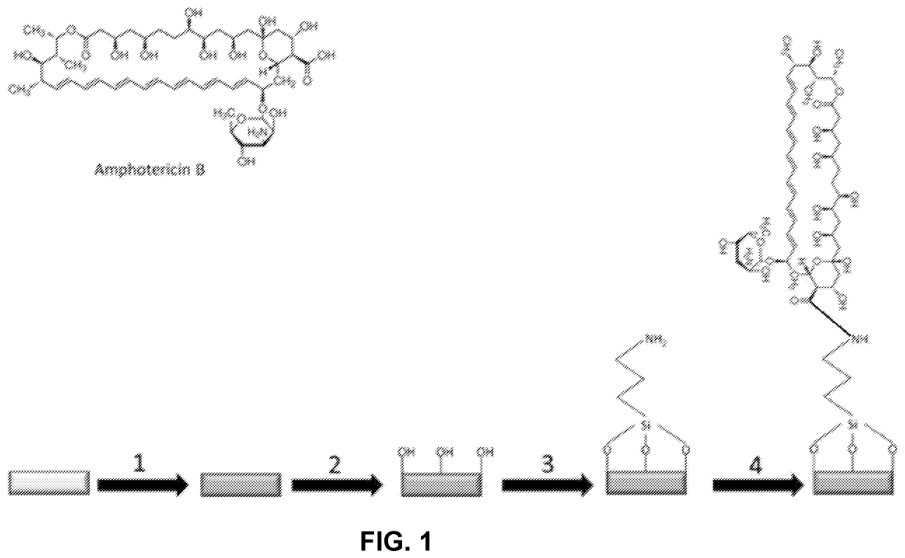

Embodiments of compositions of the present disclosure include, an anti-microbial substrate made from a medical grade polymer material and having an anti-microbial coating including a plurality of polyene antimycotic molecules coupled to the medical grade polymer on a surface of the substrate. The anti-microbial coating including the polyene antimycotic molecules is effective to treat (e.g., eliminate, reduce, and/or inhibit) or prevent fungal and bacterial growth on the surface of the substrate. In embodiments the polyene antimycotic molecules are coupled to the surface of the medical grade polymer via a linker molecule conjugated to the surface of the medical grade polymer. In embodiments, the linker molecule has free primary amines for conjugating/binding to functional groups on the polyene antimycotic molecules. In one embodiment, the linker has the formula (R¹O)₃Si—R²—NHR³ or (R¹O)₂R¹Si—R²—NHR³, where are R¹ and R² are an alkyl group, and R³ is hydrogen or an alkyl amino group. In one aspect, R¹ and R² are each C₁-C₅ alkyl groups. In one aspect, R³ is hydrogen. In another aspect, R³ is a C₁-C₅ alkyl amino group. In other embodiments, the linker molecule is aminopropyltriethoxysilane (APTMS). Other silanes with an amino functional group could also be used such as, but not limited to, the following: aminopropyltrimethoxysilane, N-2-(aminoethyl)-3-aminopropyltrimethoxysilane, N-phenyl-3-aminopropyltrimethoxysilane, N-2-(aminoethyl)-3-aminopropylmethyldimethoxysilane, etc. In embodiments, the substrate is first treated to bring/force —OH groups to the surface of the polymer material to bind with the linker molecule. In embodiments, this can be done by treating the surface of the medical grade polymer substrate with O₂ plasma, which forces —OH groups to the surface. This is illustrated in FIG. 1, steps 2 and 3.

In one aspect, the polyene antimycotic molecules are covalently bonded to the linker molecules conjugated to the surface of the medical grade polymer substrate in such a manner as to ensure that the mycosamine group of the polyene antimycotic molecule is still free to interact with/inhibit fungal cells. Since the mycosamine group cannot be used as the anchoring point for coupling the polyene antimycotic molecule to the polymer surface, in one aspect, the polyene antimycotic molecule that possesses a carboxylic acid group is pre-treated with 1-ethyl-3-(3-dimethylaminopropyl)carbodiimide (EDC) and N-hydroxysuccinimide (NHS) to convert carboxylic acid groups on the polyene antimycotic molecule to NHS-esters capable of conjugation to primary amines on the linker molecule as shown in FIG. 1, step 4 for AmB. In embodiments, Sulfo-NHS may also be used in place of NHS.

In one aspect, the anti-microbial coating comprises a plurality of units having the structure I or II covalently bonded to the substrate

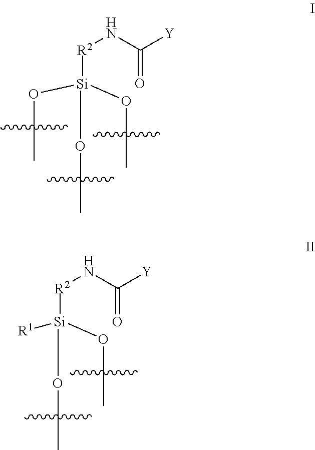

wherein R¹ and R² are an alkyl group, and
Y is a residue of the polyene antimycotic molecule.

Structure I or II is covalently bonded to the substrate via one or more Si—O bonds, where hydroxyl groups present on the surface of the substrate react with the linker (e.g., a dialkoxy or trialkoxy silane linker). Y is a residue of the polyene antimycotic molecule, which is the reaction product between the amine group in the linker and the carboxylic acid or ester thereof present in the polyene antimycotic molecule.

In one aspect, R² in structures I and II is C₁ to C₅ alkyl group. In another aspect, R² in structures I and II is a propyl group and Y is a residue of amphotericin B (AmB).

The amount of the polyene antimycotic molecules present in the anti-microbial coating can vary depending upon the application or use of the anti-microbial substrate. In one aspect, the polyene antimycotic molecules in the anti-microbial coating are in an amount of from about 0.5 nmol/cm² to about 5.0 nmol/cm², or about 0.5 nmol/cm², 1.0 nmol/cm², 1.5 nmol/cm², 2.0 nmol/cm², 2.5 nmol/cm², 3.0 nmol/cm², 3.5 nmol/cm², 4.0 nmol/cm², 4.5 nmol/cm², or 5.0 nmol/cm², where any value can be a lower and upper endpoint of a range (e.g., to 1.0 nmol/cm² to 2.5 nmol/cm²).

As described in the examples below, the anti-microbial coating of polyene antimycotic molecules on the surface of the medical grade polymer reduces growth of fungal organisms, but also appears to reduce growth of bacterial as well. However, since the polyene antimycotic molecule is tethered to the polymer surface, it does not have the same cytotoxic effects as systemic treatment with polyene antimycotic molecule that is free and not tethered. Thus, the medical grade polymers of the present disclosure can be used to reduce the incidence of fungal and bacterial infections associated with medical device use (e.g., catheters) without resorting to systemic treatment and its negative side-effects. To provide additional anti-bacterial activity, a NO-releasing agent can also be included in the medical grade polymer material. In an embodiment the medical grade polymer material is impregnated with a nitric oxide (NO) release agent. As used herein the term “impregnation” can include absorption, adsorption, swelling, covalent bonding, physical bonding, and the like. In various embodiments, the nitric oxide release agent comprises about 0.1 to about 20% by weight of the medical grade polymer, or about 1% to about 15%, or about 1% to about 10%, or about 1% to about 5%.

In an aspect, the NO-release agent releases nitric oxide (NO). In various embodiments, the nitric oxide release agent is an S-nitroso thiol of formula O═N—S—R, where R can be an alkyl or aryl moiety. Reference to alkyl and aryl moieties includes substituted and unsubstituted alkyl and aryl moieties, respectively. In an aspect, the alkyl, substituted alkyl, aryl, or substituted aryl moiety can comprise from about 5 to about 20 carbons. In an embodiment, the nitric oxide release agent may be an amino acid moiety with a thiol group. In another embodiment, the nitric oxide release agent can be an S-nitroso thiol. The S-nitroso thiol may be S-nitroso-N-acetylpenicillamine (SNAP), derivatives or salts thereof, S-Nitroso-glutathione, derivatives or salts thereof. Embodiments of the present disclosure include a treated article as above, where the NO-release agent includes an organic nitrate, a metal-NO complex, an N-nitrosamine, an S-nitrosothiol, or a combination thereof. In some aspects, the NO release agent is SNAP.

The medical grade polymer, or device made therefrom, in various embodiments, can include an elastomer. The elastomer can include a base polymer (e.g. thermoplastic polymers, thermosetting polymers, silicone, polyvinyl chloride, polyurethane, polyimide, fluoropolymer, rubber, thermoplastic elastomer). Tubing or a medical catheter, as used herein, can be any tube-shaped material, and can be formed by extrusion, heat-shrinking, or other methods. Examples of tubing include, but are not limited to, items used in medical settings such as catheters, intravenous delivery tubing, surgical tubing, drug delivery, angioplasty, neuromodulation, dilation. Tubing for on-medical applications such as food-grade tubing is also within the scope of the present disclosure.

In embodiments, the medical grade polymer material can include those with chemical resistant properties (e.g. thermoplastic elastomers, styrene-ethylene-butylene modified block copolymer with silicone oil, silicone based organic polymers, thermal set rubber, siloxane polymers (e.g, PDMS) and amorphous silica, Polypropylene-based material with USP mineral oil, ePTFE (expanded PTFE) and platinum-cured silicone, ePTFE (expanded PTFE) and fluoroelastomer, polytetrafluoroethylene, thermoplastic polyurethanes (TPU), or thermoplastic olefin elastomers (TPO)). In embodiments, the medical grade polymer material is Polydimethylsiloxane (PDMS).

Medical devices of the present disclosure and/or medical grade polymer substrates can include, but are not limited to commercially available tubing (e.g. PharMed® BPT, PureFit® SBP, PureFit® SMP, PureFit® SVP, PureFit® SWP, SaniPure™ BDF™, SaniPure™ 60, Sani-Tech® LA-60, Sani-Tech® Sil-250, Sani-Tech® STHT™-C, Sani-Tech® STHT™-R, Sani-Tech® STHT™-R-HD, Sani-Tech® STHT™-WR, Sani-Tech® STHT™-W,CO, Tygon® 2275, Tygon® 2275 I.B., Tygon® 3350, Tygon® 3355L, Tygon® 3360LA, Tygon® 3370 I.B., Tygon® LFL, Tygon® Lab (R-3603), Tygon® LFL, Tygon® Food (B-44-4X), Tygon® Fuel & Lubricant (F-4040-A), Tygon® Chemical (2001), Versilic® SPX-50, Versilic® SPX-70 I.B., Silicone (platinum-cured), Silicone (peroxide-cured), BioPharm Silicone and BioPharm Plus Silicone (platinum-cured), Puri-Flex™, C-FLEX®, PharMed® BPT, PharmaPure®, GORE® STA-PURE® PCS, GORE® STA-PURE® PFL, PTFE, Norprene® (A 60 G), Norprene® Food (A 60 F), Chem-Durance® Bio, GORE® Style 400, Viton®).

In embodiments, as mentioned above, the medical grade polymer of the present disclosure, or medical device made therefrom, is treated (e.g., impregnated) with NO-releasing material. In embodiments the medical grade polymer material is treated/impregnated with NO-releasing material prior to addition of the anti-microbial coating of polyene antimycotic molecules. In embodiments, the NO-treated medical grade polymer releases nitric oxide at a rate of from about 0.01×10⁻¹⁰ mol/min-cm² to about 4×10⁻¹⁰ mol/min-cm², or from about from about 0.05×10⁻¹⁰ mol/min-cm² to about 2×10⁻¹⁰ mol/min-cm², or from about 0.05×10⁻¹⁰ mol/min-cm² to about 2×10⁻¹⁰ mol/min-cm², or from about 0.05×10⁻¹⁰ mol/min-cm² to about 2×10⁻¹⁰ mol/min-cm², or from about 0.05×10⁻¹⁰ mol/min-cm² to about 1×10⁻¹⁰ mol/min-cm².

The surface modifications of the present disclosure can be applied to a medical grade polymers on a wide variety of medical devices including various surfaces of such devices that are associated with the cause or production of infection once administered to the subject. For example, catheters such as urinary catheters, represent a common site of infection once administered to the subject, with approximately 95% of all hospital-acquired UTI's being associated with urinary catheters and 87% of hospital-acquired bloodstream infections associated with blood vessel catheters. Thus, the surface modifications of the present disclosure can be applied to medical devices such as catheters, including surfaces such as the interior lumen and/or and exterior of catheters. The compositions and methods of the present disclosure may also be used for various other medical device applications where fungal and/or bacterial infection is prevalent when the medical device is administered to the subject such as endotracheal tubes and extracorporeal membrane oxygenation.

Embodiments of the present disclosure include medical devices made of or comprising parts made of the medical grade polymer substrates of the present disclosure described above. Ideally, medical devices or parts that will be in sustained contact with a subject (e.g., those parts in sustained contact with a subject's tissues (e.g., skin, blood, epithelium, etc.) are made of the medical grade polymer substrates of the present invention. In embodiments, medical devices of the present disclosure include catheters (e.g, urinary catheters, blood vessel catheters, etc.) or other medical tubing (e.g., endotracheal tubing, nephrostomy tubing, colostomy tubing) or medical ports, and the like. For instance, a catheter or other article of tubing is functionalized with an anti-microbial coating AmB and may optionally also be impregnated with a nitric oxide release agent. Advantageously, the treated article has low leaching of AmB.

In some aspects, the medical device of the present disclosure is a catheter or other type of medical tubing made of the medical grade polymer material and having an inner surface and an outer surface. In embodiments the inner surface is defined by an interior lumen (e.g. luminal surface or inner luminal surface). In embodiments of medical catheters/tubing of the present disclosure, the medical catheter/tubing is made of the medical grade polymer material (with the optional NO release agent) and includes the anti-microbial coating of polyene antimycotic molecules on at least the inner luminal surface of the catheter/tubing. In embodiments, the catheter/tubing of the present disclosure that includes the NO release agent, releases nitric oxide at least from the inner luminal surface of the catheter/tubing. While the catheter/tubing of the present disclosure may include the NO release agent on or near the luminal surface, the catheter/tubing could also include deeper impregnation with the NO release agent.

The present disclosure also includes methods of making the anti-microbial medical grade polymers, substrates, and devices of the present disclosure. Methods of making an embodiment of an anti-microbial, medical grade polymer substrate (and materials and devices including such substrates) includes providing or forming a medical grade polymer substrate having a surface, optionally impregnating the medical grade polymer substrate with an NO release agent and then treating the polymer surface to form the anti-microbial coating of polyene antimycotic molecules. In embodiments, forming the coating includes treating the surface of the medical grade polymer substrate with O₂ plasma to force —OH groups to the surface, then depositing a plurality of linker molecules with free primary amines on the surface such that the linker molecules conjugate to the —OH groups on the surface. Separately, a plurality of polyene antimycotic molecules are treated with 1-ethyl-3-(3-dimethylaminopropyl)carbodiimide (EDC) and N-hydroxysuccinimide (NHS) (or Sulfo-NHS) to convert carboxylic acid groups on the polyene antimycotic molecules to NHS-esters capable of conjugation to primary amines on the linker molecule. Then the plurality of EDC/NHS treated polyene antimycotic molecules are coupled to the surface of the medical grade polymer substrate via conjugation of NHS-esters of the polyene antimycotic molecules to primary amines on the linker molecules to form an anti-microbial coating on the surface of the medical grade polymer substrate. As described above, in embodiments the linker molecule can be APTMS. In embodiments where the medical grade polymer is impregnated with a NO release agent, the NO release agent can be SNAP or other NO release agent, or combination of NO release agents described above.

Aspects of the present disclosure also include using the anti-microbial, medical grade polymers of the present disclosure. In embodiments the anti-microbial, medical grade polymers are used in medical devices in order to treat and/or prevent bacterial and fungal infections typically associated with the use of such devices, such as, but not limited to, urinary catheters, blood vessel catheters, and other medical tubing and medical ports. Embodiments, include using a device of the present disclosure on or in a subject such that a portion of the device contacts the patient and the anti-microbial coating of the device (and optional NO release agent in the device material) is effective to treat and/or prevent fungal and bacterial growth on the surface of the substrate/device. In embodiments, the use of the devices of the present disclosure also treats/prevents fungal and bacterial growth on tissues of the subject contacting and/or adjacent the device.

Additional details regarding the compositions, devices, and methods of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

The following examples are put forth to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Aspects

Aspect 1. An anti-microbial substrate comprising:
a medical grade polymer material forming at least a surface of the substrate, and
an anti-microbial coating comprising a plurality of polyene antimycotic molecules coupled to the medical grade polymer on the substrate surface.
   Aspect 2. The anti-microbial substrate of Aspect 1, wherein the polyene antimycotic molecules are coupled to the surface of the medical grade polymer via a linker molecule conjugated to the surface of the medical grade polymer, the linker molecule having free primary amines.
   Aspect 3. The anti-microbial substrate of Aspect 2, wherein the linker has the formula (R¹O)₃Si—R²—NHR³ or (R¹O)₂R¹Si—R²—NHR³, where are R¹ and R² are an alkyl group, and R³ is hydrogen or an alkyl amino group.
   Aspect 4. The anti-microbial substrate of Aspect 2, wherein the linker molecule is aminopropyltriethoxysilane (APTMS), aminopropyltrimethoxysilane, N-2-(aminoethyl)-3-aminopropyltrimethoxysilane, N-phenyl-3-aminopropyltrimethoxysilane, or N-2-(aminoethyl)-3-aminopropylmethyldimethoxysilane.
   Aspect 5. The anti-microbial substrate of any one of Aspects 1-4, wherein the polyene antimycotic molecules comprises amphotericin B (AmB), nystatin, natamycin, or hamycin.
   Aspect 6. The anti-microbial substrate of any one of Aspects 1-5, wherein the medical grade polymer comprises a thermoplastic polymer, a thermosetting polymer, silicone, polyvinyl chloride, polyurethane, polyimide, fluoropolymer, rubber, or a thermoplastic elastomer.
   Aspect 7. The anti-microbial substrate of any one of Aspects 1-6, wherein the medical grade polymer material is impregnated with a nitric oxide (NO) release agent.
   Aspect 8. The anti-microbial substrate of Aspect 7, wherein the NO release agent is an S-nitroso thiol of formula O═N—S—R, wherein R is an alkyl moiety or aryl moiety.
   Aspect 9. The anti-microbial substrate of Aspect 7, wherein the NO release agent is S-nitroso-N-acetylpenicillamine (SNAP), S-nitroso-glutathione, and S-nitroso-N-acetylcysteine.
   Aspect 10. The anti-microbial substrate of any one of Aspects 7-9, wherein the NO release agent comprises about 0.1 to about 20% by weight of the medical grade polymer.
   Aspect 11. The anti-microbial substrate of any one of Aspects 7-10, wherein the medical grade polymer releases nitric oxide at a rate of from about 0.01×10⁻¹⁰ mol/min-cm² to about 4×10⁻¹⁰ mol/min-cm².
   Aspect 12. The anti-microbial substrate of any one of Aspects 1-11, wherein the polyene antimycotic molecules are in an amount of from about 0.5 nmol/cm² to about 5.0 nmol/cm².
   Aspect 13. The anti-microbial substrate of any one of Aspects 1-12, wherein the anti-microbial coating comprises a plurality of units having the structure I covalently bonded to the substrate

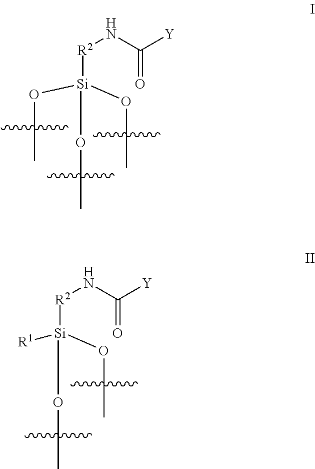

wherein R¹ and R² is an alkyl group, and
Y is a residue of the polyene antimycotic molecule.
   Aspect 14. The anti-microbial substrate of Aspect 13, wherein R² is C₁ to C₅ alkyl group.
   Aspect 15. The anti-microbial substrate of Aspect 13, wherein R² is a propyl group and Y is a residue of amphotericin B (AmB).
   Aspect 16. The anti-microbial substrate of any one of Aspects 13-15, wherein the medical grade polymer material is impregnated with a nitric oxide (NO) release agent.
   Aspect 17. The anti-microbial substrate of Aspect 16, wherein the NO release agent is S-nitroso-N-acetylpenicillamine (SNAP), S-nitroso-glutathione, and S-nitroso-N-acetylcysteine.
   Aspect 18. The anti-microbial substrate of Aspect 13, wherein R² is a propyl group, Y is a residue of amphotericin B (AmB), and the medical grade polymer material is impregnated with S-nitroso-N-acetylpenicillamine (SNAP).
   Aspect 19. A medical device comprising the anti-microbial substrate of any of Aspects 1-18.
   Aspect 20. The medical device of Aspect 19, wherein the medical device is a catheter or medical tubing comprising an interior lumen having a luminal surface and wherein the luminal surface comprises the anti-microbial coating.
   Aspect 21. The medical device of Aspect 20, wherein the catheter or medical tubing is selected from the group consisting of: urinary catheters, blood vessel catheters, endotracheal tubing, nephrostomy tubing, colostomy tubing, and medical ports.
   Aspect 22. A method of making an anti-microbial, medical grade polymer substrate, the method comprising:
providing or forming a medical grade polymer substrate having a surface;
treating the surface of the medical grade polymer substrate with O₂ plasma to force —OH groups to the surface;
depositing a plurality of linker molecules with free primary amines on the surface such that the linker molecules conjugate to the —OH groups on the surface;
treating a plurality of polyene antimycotic molecules with 1-ethyl-3-(3-dimethylaminopropyl)carbodiimide (EDC) and N-hydroxysuccinimide (NHS) to convert carboxylic acid groups on the polyene antimycotic molecules to NHS-esters capable of conjugation to primary amines on the linker molecule; and
coupling the plurality of EDC/NHS treated polyene antimycotic molecules to the surface of the medical grade polymer substrate via conjugation of NHS-esters of the polyene antimycotic molecules to primary amines on the linker molecules to form an anti-microbial coating on the surface of the medical grade polymer substrate.
   Aspect 23. The method of Aspect 22, further comprising impregnating the medical grade polymer substrate with a nitric oxide (NO) release agent.
   Aspect 24. The method of Aspect 23, wherein impregnating the medical grade polymer substrate with the NO release agent is performed before treating the surface of the medical grade polymer substrate with O₂ plasma.
   Aspect 25. The method of Aspect 23, wherein the NO release agent is an S-nitroso thiol of formula O═N—S—R, wherein R is an alkyl moiety or aryl moiety.
   Aspect 26. The method of Aspect 25, wherein the S-nitroso thiol is selected from the group consisting of: S-nitroso-N-acetylpenicillamine (SNAP), S-nitroso-glutathione, and S-nitroso-N-acetylcysteine.
   Aspect 27. A method of treating or preventing a fungal and bacterial infection in a subject associated with a medical device, the method comprising: administering the medical device of any one of Aspects 19-21 to the subject, such that a portion of the device contacts the subject, wherein the anti-microbial coating treats or prevents fungal and bacterial growth on the surface of the substrate and/or on a tissue of the subject adjacent to the device.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1—Amphotericin B and/or SNAP Immobilization to Medical Grade Polymers

The present example describes fabrication of a dual antibacterial/antifungal surface, preparation of the surface in a two-step process, and demonstrates its effectiveness against common nosocomial pathogens with and without the addition of the NO donor S-nitroso-acetylpenicillamine (SNAP). The addition of the NO donor SNAP was included in order to represent that the AmB immobilization can also be combined with antibacterial release mechanisms. AmB was immobilized through an EDC/NHS coupling method. Samples SNAP-AmB materials were first swelled with SNAP prior to AmB immobilization. We confirmed successful AmB immobilization to a medical grade polymer through an amine quantification assay and alterations in contact angles and demonstrated the efficacy of AmB-immobilized surfaces with and without an additional antibacterial agent against fungal and bacterial pathogens. This novel, broad-spectrum approach was the first to immobilize an antifungal agent to a medical grade polymer and combine antibacterial, NO-releasing technology with an immobilized antifungal agent, which is capable of reducing the rate of infection associated with indwelling medical devices

### Materials and Methods

### SNAP Synthesis

The method for the synthesis of SNAP was adapted via a previously defined method described in Chipinda, et al., J Phys Chem B 2006 (reference 14, hereby incorporated by reference herein for synthesis of SNAP). Briefly, a 1:1 molar ration of NaNO₂ and NAP was added to a solution of DI water and methanol containing 2 M HCl and 2 M H₂SO₄ and stirred for 30 min. The reaction vessel was then transferred to an ice bath and cooled for 6 h until SNAP crystals precipitated. SNAP crystals were collected via vacuum filtration and dried in a desiccator for 24 h to remove any trace solvent. For the duration of this procedure, the reaction mixture and products were sheltered from light.

### Preparation of SNAP-Impregnated PDMS

A 25 mg/mL SNAP swelling solution was prepared by dissolving SNAP in THF according to previously optimized NO release kinetics as described in Brisbois, et al., Acta Biomater 2016 and Goudie, et al, Sci Rep 2017 (References 15 and 16, both of which are hereby incorporated by reference herein for incorporation of SNAP). The prepared PDMS was added to the SNAP-THF solution for 24 h. After swelling, the PDMS was removed, briefly washed in PBS, and dried overnight in the dark at room temperature to allow any excess THF to evaporate. After drying, the samples were immersed in DI water and sonicated for 5 min to remove any SNAP crystals from the surface.

Amphotericin B (AmB) Immobilization

The antifungal molecule AmB was immobilized onto PDMS substrates using EDC/NHS coupling as illustrated in FIG. 1. Optionally, prior to immobilization of AmB, the PDMS can be impregnated with SNAP as described above and shown in step 1. EDC/NHS coupling was chosen as the coupling method due to it being designed for the immobilization of large biomolecules.¹⁷ Prior to EDC/NHS coupling, surfaces were treated with O₂ plasma to force —OH groups on the surface (FIG. 1, step 2), which allows for chemical vapor deposition of APTMS. Once deposited, APTMS formed a layer of primary amines on the surface of the silicone rubber, which allows for EDC/NHS coupling (FIG. 1, step 3). Once the aminated surface was formed, the EDC/NHS coupling reaction was mixed to allow for proper priming before immobilization onto the surface. EDC reacts with carboxylic acid groups present on AmB, which is then replaced by NHS to form an ester that is considerably more stable than the EDC intermediate, allowing for more efficient conjugation to primary amines. After 10 minutes passed for the AmB—NHS complex to form, the aminated PDMS substrates were subjected to the reaction mixture to allow for AmB immobilization to the surface (FIG. 1, step 4).

### Results and Discussion

Material Characterization

Fabrication of SNAP-AmB PDMS polymer. This example provides proof-of-concept that antifungal agents can be immobilized in a similar manner as antibacterial agents.¹⁸ This is important to establish as, despite the increased cytotoxicity of antifungal agents when compared to antibacterial agents, there is scarce literature available on the localized immobilization of antifungal agents. For this study, AmB was chosen as a model antifungal agent as it is considered an essential medicine by the World Health Organization and is readily available as a generic medication worldwide.¹⁹ AmB works by binding to ergosterol (the main sterol of fungal cells) in the cell membrane and has three proposed mechanisms of action: 1) AmB molecules aggregate to form an ion-channel “pore” in the cell membrane, which causes leakage of intracellular components. 2) AmB causes oxidative stress by binding to low-density lipoprotein receptors. 3) AmB molecules adsorb onto the cell membrane which destabilizes the cell membrane by sequestering ergosterol.²⁰ If the density of immobilized AmB is high enough, then the pores proposed in the first mechanism should be capable of forming; however, the latter two proposed mechanisms should be feasible regardless of the density of surface AmB. The mycosamine group on AmB allows it to bind to sterols, and cannot bind to sterols without it; therefore, any coupling method cannot use the mycosamine group as an anchoring point. With that in mind, the EDC/NHS reaction was chosen as the coupling method, as it allows for the carboxylic group to be used as the anchoring point.¹⁷

In addition to the immobilization of AmB, NO-releasing technology was utilized in an attempt to create a broad-spectrum material capable of exhibiting antibacterial properties while promoting hemocompatibility (in addition to AmB's antifungal property). Previous studies have optimized a SNAP solvent swelling protocol, demonstrating that a 25 mg/mL SNAP-THF swelling solution (1) allows for rapid solvent evaporation after swelling, (2) has excellent solvent swelling behavior, (3) optimizes NO release kinetics, (4) minimizes the volumes of solution required, and (5) can be used after the polymer extrusion process (which limits depletion of NO reservoir due to the high heat used during extrusion).¹⁵⁻¹⁶

The immobilization of AmB and implementation of NO-releasing technology was carried out through a two-step synthesis process. PDMS was swelled in a 25 mg/mL SNAP-THF solution for 24 h and dried under dark ambient conditions overnight to allow excess THF to evaporate. Prior to EDC/NHS coupling of AmB, SNAP PDMS was surface treated with O₂ plasma to force —OH groups onto the surface of the PDMS. A layer of primary amines was formed on the surface of the PDMS via chemical vapor deposition of APTMS. Prior to immobilization, an EDC/NHS reaction was performed for 10 minutes. In this reaction, the carboxylic acid groups present on AmB are converted to NHS-esters, allowing for more efficient conjugation to primary amines.¹⁷ After formation of the AmB—NHS ester, aminated samples were placed within the reaction vessel, and AmB was immobilized onto the PDMS surface. The following sections characterize the physical and biological properties of the fabricated SNAP-AmB PDMS polymer.

Amphotericin surface quantification. AmB immobilization was confirmed by using an amine quantifying assay. The ninhydrin assay has been widely used for both qualitative²¹ and quantitative²² determination of amines in peptides and proteins and also has been applied to quantify the presence of amines group on nanomaterials and thin films surfaces, including silica NPs, carbon nanotubes and planar silica surfaces.²³⁻²⁶ The reaction of primary amines with ninhydrin occurs by an initial nucleophilic displacement of a ninhydrin hydroxyl group by amine, followed by a complex mechanism that generates a colored product known as Ruhemann's purple.²³ The generation of free dye in solution for colorimetric analysis has the advantage of being less likely to suffer from steric effects when quantifying high surface coverages of amine. Initial studies used a literature procedure in ethanol solvent and the reported extinction coefficient for the product dye to assess suitability of the assay.²⁷ Therefore, in our study, AmB immobilized-PDMS with a uniform diameter and thickness was used to quantify the functional amine group to correspond the amount of drug loaded into the PDMS polymer through a ninhydrin assay. As shown in Table 1, both SNAP and SNAP-AmB samples showed amines present, with SNAP-AmB showing significantly higher concentrations of amine per well (p<0.05). This is expected as both SNAP and AmB molecules contain amine groups. Therefore, we can conclude from these results that both the swelling of SNAP and the immobilization of AmB onto the medical grade polymer was successful. It should be noted that untreated PDMS resulted in a reading of 0, which is due to PDMS not having an amine group and meaning the assay was performed correctly.

TABLE 1

| Sample Type | Final Concentration (μM) per well |
|---|---|
| PDMS | 0.00 ± 0.0 |
| SNAP | 76.5 ± 16.9 |
| SNAP-AmB | 677.8 ± 12.6 |

Quantification of amines present in fabricated materials. Amines present in both the SNAP and AmB molecules contribute to the total concentration of amine found in the materials. Measurements are reported in mean ± SD.

It was determined that the PDMS surface contained 1.92±0.45 nmol AmB cm⁻² (n=8). EDS imaging revealed the AmB surface is uniformly present on the silicone rubber surface.

Contact Angle Measurements

After confirmation of AmB immobilization, the contact angle of samples throughout the two-step synthesis process was measured in order to assess the effect on the surface wettability of PDMS. Each step in the synthesis process had an effect on surface wettability. PDMS exhibited a hydrophobic contact angle of 110.54°±2.19, which was only slightly affected by the inclusion of SNAP in SNAP PDMS samples (105.56°±2.32). This slight reduction is due to the presence of nitrogen and oxygen atoms capable of hydrogen bonding in the SNAP molecule. The addition of the aminated surface through AMPTS immobilization significantly affected sample surface wettability and made the surface of both aminated PDMS and aminated SNAP PDMS samples slightly hydrophilic (79.04°±2.17 and 80.50°±1.86, respectively), which is due to the primary amine on AMPTS being capable of forming hydrogen bonds with water. An even greater increase in hydrophilicity was seen with the immobilization of AmB as the contact angle decreased to 59.40°±2.71 and 59.54°±2.57 on AmB PDMS and AmB SNAP PDMS, respectively. This result is expected as the polyol subunit of AmB (which contains multiple —OH groups) is also capable of hydrogen bonding with water.²⁰

Measurements of NO Release

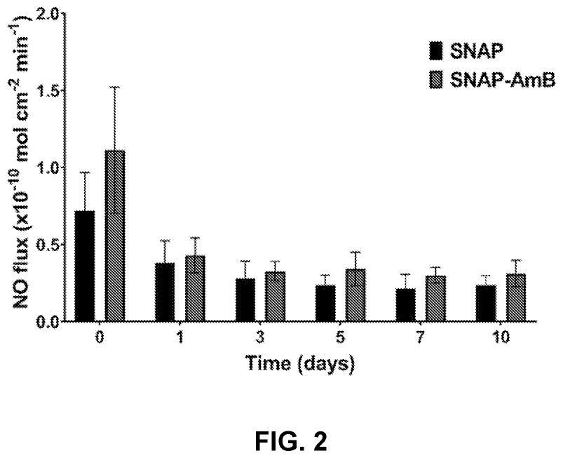

Nitric oxide release measurements. NO-releasing materials have previously demonstrated a reduction in the viability of a wide number of microbes and a limitation in platelet activation,²⁸⁻²⁹ but few materials have demonstrated extended NO-releasing capabilities for long-term (>24 h) applications. Previous studies have routinely demonstrated the catalytic, stable release of NO from S-nitrosothiols (including SNAP) impregnated in polymers by exposure to heat, light irradiation, moisture, or metal ions.³⁰⁻³² Therefore, in this study, PDMS, a polymer commonly used for blood-contacting medical devices including catheters, implants, and pacemaker encapsulants, was swelled with the NO donor SNAP, and NO release kinetics were measured at 37° C. in PBS to mimic physiological conditions over 10 days (FIG. 2). Samples were swelled with a previously optimized concentration of 25 mg/mL SNAP concentration in THF due to (1) its high vapor pressure, allowing for rapid solvent evaporation after swelling and (2) its excellent solvent swelling behavior.¹⁵⁻¹⁶ Further, the effect of the immobilization of the antifungal agent Amphotericin B on the NO release profile was evaluated. The overall NO release profile for both the SNAP and SNAP-AmB PDMS samples showed consistent, moderate levels of NO release within a range previously shown to be effective in improving the hemocompatibility and antibacterial activity in short-term and long-term medical device applications.^{15, 33} The initial NO flux for the SNAP and SNAP-AmB PDMS samples (0.83±0.32×10⁻¹⁰ mol cm⁻² min⁻¹ and 0.84±0.23×10⁻¹⁰ mol cm⁻² min⁻¹, respectively) were not significantly different (p>0.05). Both SNAP and SNAP-AmB showed a stabilized, consistent flux for 10 days, displaying a flux of 0.24±0.10×10⁻¹⁰ mol cm⁻² min⁻¹ and 0.32±0.10×10⁻¹⁰ mol cm⁻² min⁻¹, respectively, by Day 10. Similar NO release rates for SNAP-incorporated polymers have been established in literature to be effective for reducing platelet adhesion and increasing antimicrobial properties of medical-grade polymers.^{16, 33}

It should be noted that although the SNAP-AmB samples showed a slightly higher NO flux over the 10 days of measurement compared to SNAP samples, the difference was not significant (p>0.05) with the exception on Day 7. The slightly increased NO flux may be attributed to the increased hydrophilicity of the SNAP-AmB surface (confirmed by the contact angle characterization previously discussed), which can improve water uptake and therefore increase the NO release rate. Similar effects due to hydrophilicity have been previously noted in literature.³⁴⁻³⁵ However, because the difference is not significant, this demonstrates that the addition of AmB does not negatively alter the NO release profile over a prolonged period of time.

To ensure that the AmB immobilization step did not adversely affect the SNAP reservoir, the total SNAP loading of samples before and after AmB immobilization was measured. SNAP loading was measured by placing freshly made samples within THF, and then allowing the SNAP reservoir to leach out over a 5 h period. Thereafter, SNAP concentration was measured with a UV-vis spectrometer and interpolated with a calibration curve. The results revealed that the AmB immobilization process had no effect on the SNAP reservoir, as there was no statistically significant difference between samples with or without AmB (2.58±0.36 mg and 2.41±0.23 mg respectively). SNAP leaching in physiological conditions was not measured for this study, as previously reported hydrophilic biomacromolecules immobilized onto SNAP-swelled surfaces did not show an increase in SNAP leaching

Antimicrobial Activity

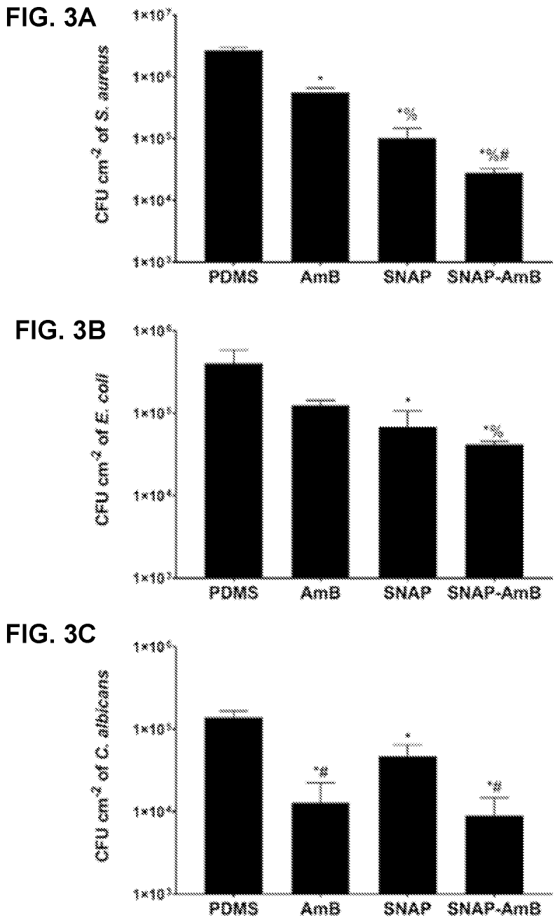

To date, few studies have demonstrated simultaneous broad antimicrobial efficacy against clinically relevant bacterial and fungal pathogens. NO-releasing materials have demonstrated tremendous promise for reducing the viability of numerous bacterial strains with minimal resistance,³⁷⁻³⁸ but have had little success against fungal pathogens, which requires much higher concentration of NO to be effective.⁹⁻¹¹ Therefore, with an aim to increase the antimicrobial activity of polymers used for medical devices, the antifungal agent Amphotericin B was immobilized onto the surface of SNAP-infused PDMS. To assess the antimicrobial efficacy of the synthesized materials, a series of 24 h in vitro antimicrobial assays were performed to assess the viability of adhered bacteria and fungi commonly associated with hospital-acquired infections after exposure to the materials. Isolated strains of S. aureus (Gram-positive bacteria), E. coli (Gram-negative bacteria), and C. albicans (opportunistic fungi) were exposed to each sample type for 24 h at 37° C. at 150 rpm. FIGS. 3A-3C summarize the effects of the different surface modifications on the viability of the adhered pathogens: S. aureus (FIG. 3A), E. coli (FIG. 3B), and C. albicans (FIG. 3C). As demonstrated, AmB-immobilized surfaces significantly reduced the viability of adhered C. albicans compared to control PDMS surfaces (AmB—90.8±7.0%; SNAP-AmB—93.5±4.2%; p<0.05) (FIG. 3C). Amphotericin B binds to ergosterol present in the cell membrane of fungi, which serves a similar role to that of cholesterol in mammalian cells, causing membrane destabilization.³⁹ After binding, the leakage of monovalent ions as a result of the formation of ion channels results in membrane depolarization.⁴⁰ The presence of AmB can also result in oxidative damage and mitochondrial disruption, although the exact mechanism of this is still unknown.³⁹ Although samples with only SNAP infused were able to reduce the viability of adhered C. albicans, the difference in reduction was not as great (65.8±12.5%). Although nitric oxide-generating surfaces have previously shown some success in reducing adhered viable C. albicans, reduced efficacy of NO against fungi relative to bacteria results in the need to combine NO-releasing devices with antifungal strategies to reduce the risk of polymicrobial infections.⁴¹ C. albicans has been shown to have more robust adherence capacity than other Candidad species.⁵⁶

NO-releasing surfaces have been shown previously to reduce bacterial viability through several different mechanisms including DNA cleavage, nitrosative and oxidative stress, and peroxynitrite or superoxide formation.³¹ As expected, SNAP surfaces significantly reduced the viability of adhered S. aureus and E. coli compared to PDMS controls surfaces (SNAP vs. S. aureus—96.2±1.6%; SNAP vs. E. coli—83.2±9.3%; SNAP-AmB vs. S. aureus—99.0±0.2%; SNAP-AmB vs. E. coli—89.7±1.0%; p<0.05). The presence of AmB did not interfere with the antibacterial nature of NO-releasing surfaces. In fact, the presence of AmB significantly reduced the number of viable adhered S. aureus and E. coli by 79.4±3.8% and 69.5±4.7%, respectively (p<0.05). As discussed previously, AmB immobilization resulted in a reduced contact angle, making the surface more hydrophilic. Hydrophilic surfaces with similar contact angles have exhibited similar reductions in adhered bacterial pathogens.⁴²⁻⁴³ SNAP-AmB surfaces best reduced the number of viable S. aureus (99.0±0.2%) and E. coli (89.7±1.0%) on the surfaces (p<0.05). These results are consistent with other NO-releasing materials, which have similarly reduced the viability of both Gram-positive and Gram-negative bacterial pathogens by >80%.³⁰⁻³³ The resulting antifungal-immobilized NO-releasing surface modification can be applied to limitless medical-grade polymers, demonstrating the potential of the surface's dual-antimicrobial functionality for biomedical applications

Antiplatelet Activity

Currently, the systemic administration of anticoagulant or antiplatelet therapies are the clinical standard in preventing medical device-induced clot formation. However, regardless of the therapeutic agent used, the most common adverse side effect from systemic anticoagulation is acute hemorrhaging including gastrointestinal and intracranial bleeding.⁴⁴ Due to these complications, the systemic administration of anticoagulation leads the United States in clinical drug-related deaths.⁴⁵⁻⁴⁶ Therefore, a surface modification which prevents platelet adhesion and activation is of high demand.

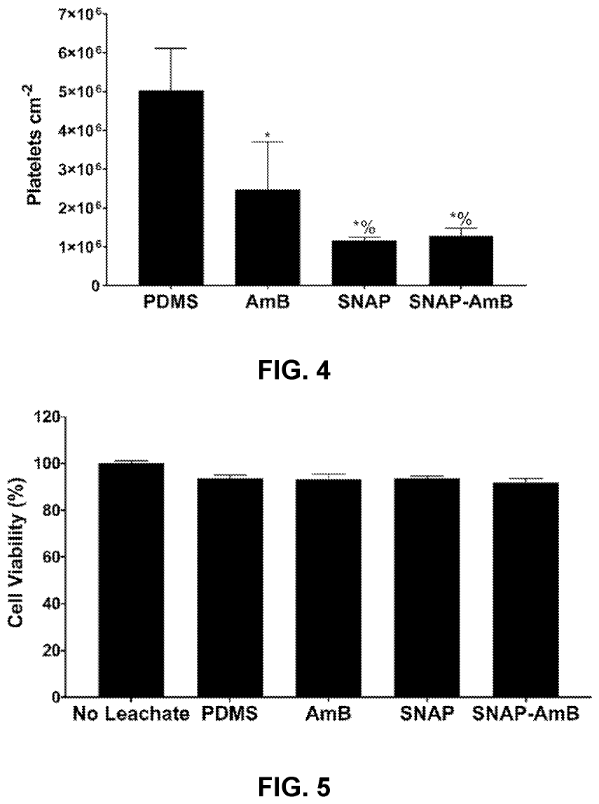

In this study, the antiplatelet activity of the fabricated materials was measured using an LDH assay after in vitro exposure to porcine plasma (2×10⁸ platelets/mL) for 1.5 h (FIG. 4). Both SNAP and AmB SNAP samples exhibited a potent antiplatelet effect with a respective reduction of 76.93±1.76% and 74.57±3.90%. This reduction can be attributed to the release of NO from the surface of the samples. The endothelium of blood vessels passively releases NO into the bloodstream at an estimated flux of 0.5−4×10⁻¹⁰ mol cm⁻² min⁻¹ in order to prevent platelet activation and adhesion;⁴⁷⁻⁴⁸ therefore, it was expected that NO-releasing samples would result in platelet reduction due to having a NO flux within that range (FIG. 5). Surprisingly, AmB PDMS samples resulted in a 50.91±24.45% reduction of platelets compared to PDMS, which was statistically significant. This reduction can be attributed to the surface hydrophilicity due to AmB immobilization, which can also exhibit antifouling behavior.⁴⁹

Hemolysis

Erythrocytes can be lysed via the exposure of foreign materials due to contact, toxins, metal ions, leachates, and surface charge, leading to decreased oxygen transport, toxicity, and altered kidney function.⁵⁰⁻⁵¹ To measure the hemolytic activity of the fabricated materials, samples (n=6) were incubated directly in vitro with whole porcine blood for 2 h at 37° C. according to ISO 10993-4 protocol (Table 2). All fabricated materials were found to be nonhemolytic (hemolytic index <2%), and the positive control (sterile DI water) had a hemolytic index ˜100%. No statistical difference was found between the sample types (p>0.05). Thus, all fabricated materials were found to be safe according to a 2 h in vitro whole blood study.

TABLE 2

| Sample Type | Hemolysis (%) |
|---|---|
| PDMS | 0.49 ± 0.54 |
| AmB | 0.65 ± 1.18 |
| SNAP | 0.16 ± 0.40 |
| SNAP-AmB | 0.00 ± 0.00 |
| Sterile DI water | 96.06 ± 4.31 |

Hemolytic activity of PDMS, AmB, SNAP, and SNAP-AmB materials. All materials were found to be non-hemolytic (<2%). Sterile DI water was used as the positive control.

Cytotoxicity

Investigating cytotoxicity is a necessary preliminary biological evaluation in determining the toxicity of different materials towards host cells. In this study, the toxicity of PDMS, AmB, SNAP, and SNAP-AmB materials were measured using cytotoxicity testing against human fibroblasts in accordance with ISO 10993 standards. Leachates from the materials were collected after incubating samples in EMEM at 37° C. for 24 h and exposed to human fibroblasts prepared in a 96-well plate. Control wells were also prepared with human fibroblasts exposed to no leachates. After 24 h of exposure to leachates, a CCK-8 dye was added, and after 3 h of additional incubation, the color intensity was measured at 450 nm using a plate reader. This colorimetric assay measures the reduction of a WST-8 salt by cellular dehydrogenases, resulting in an orange formazan product, which corresponds to the number of living cells. With respect to control human fibroblasts, no cytotoxicity (>90% human fibroblast viability) was measured from any of the materials (PDMS—93.4±1.4%; AmB—93.2±2.2%; SNAP—93.4±1.1%; and SNAP-AmB—91.8±1.7%). No significant difference was found between any of the sample types (p>0.05). Previous studies have reported similar non-cytotoxic behavior from other SNAP-based materials.^{31-32, 52-53} Amphotericin B dosages must be monitored as high concentrations may lead to local and systemic toxicity.⁵⁴ However, the concentration of Amphotericin B present in the leachates in this study did not adversely affect fibroblast viability. Given the promising in vitro cytotoxicity results from this study, further in vivo testing is should be performed to dictate the safety of the fabricated materials.

### CONCLUSIONS

The findings from this study provide a promising platform for combatting polymicrobial colonization and improving the hemocompatibility of polymer surfaces. In this study, NO-releasing Amphotericin B-immobilized PDMS was developed using a two-step solvent swelling and EDC/NHS coupling method. The synthesized materials demonstrated consistent a NO flux over 10 days with minimal leaching. Surface functionalization with Amphotericin B reduced the contact angle from ca. XX° (untreated PDMS, SNAP) to XX° (AmB, SNAP-AmB). Further, amine quantification showed increased amine content for SNAP-AmB (XX) and AmB samples (XX) compared to SNAP (XX) and untreated PDMS). The resulting surface modifications significantly decreased the viability of adhered S. aureus (99.0±0.2%), E. coli (89.7±1.0%), and C. albicans (93.5±4.2%), presenting an all-encompassing alternative method for unilaterally combatting polymicrobial infections as opposed to current standards of care. The NO-releasing surfaces improved antiplatelet activity (ca. 75% reduction in platelet adhesion) compared to untreated surfaces. None of the surface modifications developed in this study resulted in cytotoxic effects towards human fibroblasts (<90% cell viability), and no hemolytic activity (<2% hemolysis) was found. This study presents a proof of concept for a singular-platform solution that simultaneously combats polymicrobial infection and prevents surface-induced thrombosis.

### REFERENCES

1. Kojic, E. M.; Darouiche, R. O., Candida infections of medical devices. Clin Microbiol Rev 2004, 17 (2), 255-67.
2. Cepas, V.; Lopez, Y.; Munoz, E.; Rolo, D.; Ardanuy, C.; Marti, S.; Xercavins, M.; Horcajada, J. P.; Bosch, J.; Soto, S. M., Relationship Between Biofilm Formation and Antimicrobial Resistance in Gram-Negative Bacteria. Microb Drug Resist 2019, 25 (1), 72-79.
3. Nguyen, N. T.; Grelling, N.; Wetteland, C. L.; Rosario, R.; Liu, H., Antimicrobial Activities and Mechanisms of Magnesium Oxide Nanoparticles (nMgO) against Pathogenic Bacteria, Yeasts, and Biofilms. Sci Rep 2018, 8 (1), 16260.
4. Romling, U.; Kjelleberg, S.; Normark, S.; Nyman, L.; Uhlin, B. E.; Akerlund, B., Microbial biofilm formation: a need to act. J Intern Med 2014, 276 (2), 98-110.
5. Coad, B. R.; Griesser, H. J.; Peleg, A. Y.; Traven, A., Anti-infective Surface Coatings: Design and Therapeutic Promise against Device-Associated Infections. PLoS Pathog 2016, 12 (6), e1005598.
6. Jabra-Rizk, M. A.; Kong, E. F.; Tsui, C.; Nguyen, M. H.; Clancy, C. J.; Fidel, P. L.; Noverr, M., Candida albicans Pathogenesis: Fitting within the Host-Microbe Damage Response Framework. Infect Immun 2016, 84 (10), 2724-2739.
7. Roscetto, E.; Contursi, P.; Vollaro, A.; Fusco, S.; Notomista, E.; Catania, M. R., Antifungal and anti-biofilm activity of the first cryptic antimicrobial peptide from an archaeal protein against Candida spp. clinical isolates. Sci Rep 2018, 8 (1), 17570.
8. Vargas-Blanco, D.; Lynn, A.; Rosch, J.; Noreldin, R.; Salerni, A.; Lambert, C.; Rao, R. P., A pre-therapeutic coating for medical devices that prevents the attachment of Candida albicans. Ann Clin Microb Anti 2017, 16.
9. Ghaffari, A.; Miller, C. C.; McMullin, B.; Ghahary, A., Potential application of gaseous nitric oxide as a topical antimicrobial agent. Nitric Oxide 2006, 14 (1), 21-9.
10. Macherla, C.; Sanchez, D. A.; Ahmadi, M. S.; Vellozzi, E. M.; Friedman, A. J.; Nosanchuk, J. D.; Martinez, L. R., Nitric oxide releasing nanoparticles for treatment of Candida albicans burn infections. Front Microbiol 2012, 3, 193.
11. Stasko, N.; McHale, K.; Hollenbach, S. J.; Martin, M.; Doxey, R., Nitric Oxide-Releasing Macromolecule Exhibits Broad-Spectrum Antifungal Activity and Utility as a Topical Treatment for Superficial Fungal Infections. Antimicrob Agents Chemother 2018, 62 (7).
12. Boogaerts, M.; Winston, D. J.; Bow, E. J.; Garber, G.; Reboli, A. C.; Schwarer, A. P.; Novitzky, N.; Boehme, A.; Chwetzoff, E.; De Beule, K.; Itraconazole Neutropenia Study, G., Intravenous and oral itraconazole versus intravenous amphotericin B deoxycholate as empirical antifungal therapy for persistent fever in neutropenic patients with cancer who are receiving broad-spectrum antibacterial therapy. A randomized, controlled trial. Ann Intern Med 2001, 135 (6), 412-22.
13. Laniado-Laborin, R.; Cabrales-Vargas, M. N., Amphotericin B: side effects and toxicity. Rev Iberoam Micol 2009, 26 (4), 223-7.
14. Chipinda, I.; Simoyi, R. H., Formation and stability of a nitric oxide donor: S-nitroso-N-acetylpenicillamine. J Phys Chem B 2006, 110 (10), 5052-61.
15. Brisbois, E. J.; Major, T. C.; Goudie, M. J.; Bartlett, R. H.; Meyerhoff, M. E.; Handa, H., Improved hemocompatibility of silicone rubber extracorporeal tubing via solvent swelling-impregnation of S-nitroso-N-acetylpenicillamine (SNAP) and evaluation in rabbit thrombogenicity model. Acta Biomater 2016, 37, 111-9.
16. Goudie, M. J.; Pant, J.; Handa, H., Liquid-infused nitric oxide-releasing (LINORel) silicone for decreased fouling, thrombosis, and infection of medical devices. Sci Rep 2017, 7 (1), 13623.
17. Fischer, M. J., Amine coupling through EDC/NHS: a practical approach. In Surface plasmon resonance, Springer: 2010; pp 55-73.
18. Kallenberg, A. I.; van Rantwijk, F.; Sheldon, R. A., Immobilization of penicillin G acylase: the key to optimum performance. Advanced Synthesis & Catalysis 2005, 347 (7-8), 905-926.
19. Model List of Essential Medicines; World Health Organization: 2019.
20. Kamiński, D. M., Recent progress in the study of the interactions of amphotericin B with cholesterol and ergosterol in lipid environments. European biophysics journal: EBJ 2014, 43 (10-11), 453-467.
21. Kaiser, E.; Colescott, R. L.; Bossinger, C. D.; Cook, P. I., Color test for detection of free terminal amino groups in the solid-phase synthesis of peptides. Anal Biochem 1970, 34 (2), 595-8.
22. Sarin, V. K.; Kent, S. B.; Tam, J. P.; Merrifield, R. B., Quantitative monitoring of solid-phase peptide synthesis by the ninhydrin reaction. Anal Biochem 1981, 117 (1), 147-57.
23. Friedman, M., Applications of the ninhydrin reaction for analysis of amino acids, peptides, and proteins to agricultural and biomedical sciences. J Agric Food Chem 2004, 52 (3), 385-406.
24. Hristov, D. R.; Rocks, L.; Kelly, P. M.; Thomas, S. S.; Pitek, A. S.; Verderio, P.; Mahon, E.; Dawson, K. A., Tuning of nanoparticle biological functionality through controlled surface chemistry and characterisation at the bioconjugated nanoparticle surface. Sci Rep 2015, 5, 17040.
25. Lu, H. T., Synthesis and characterization of amino-functionalized silica nanoparticles. Colloid Journal 2013, 75 (3), 311-318.
26. Sun, Y.; Kunc, F.; Balhara, V.; Coleman, B.; Kodra, O.; Raza, M.; Chen, M.; Brinkmann, A.; Lopinski, G. P.; Johnston, L. J., Quantification of amine functional groups on silica nanoparticles: a multi-method approach. Nanoscale Advances 2019, 1 (4), 1598-1607.
27. Iannazzo, D.; Piperno, A.; Ferlazzo, A.; Pistone, A.; Milone, C.; Lanza, M.; Cimino, F.; Speciale, A.; Trombetta, D.; Saija, A.; Galvagno, S., Functionalization of multi-walled carbon nanotubes with coumarin derivatives and their biological evaluation. Org Biomol Chem 2012, 10 (5), 1025-31.
28. Gierke, G. E.; Nielsen, M.; Frost, M. C., S-Nitroso-N-acetyl-D-penicillamine covalently linked to polydimethylsiloxane (SNAP-PDMS) for use as a controlled photoinitiated nitric oxide release polymer. Sci Technol Adv Mater 2011, 12 (5), 055007.
29. Pant, J.; Gao, J.; Goudie, M. J.; Hopkins, S. P.; Locklin, J.; Handa, H., A multi-defense strategy: Enhancing bactericidal activity of a medical grade polymer with a nitric oxide donor and surface-immobilized quaternary ammonium compound. Acta Biomater 2017, 58, 421-431.
30. Douglass, M. E.; Goudie, M. J.; Pant, J.; Singha, P.; Hopkins, S.; Devine, R.; Schmiedt, C. W.; Handa, H., Catalyzed Nitric Oxide Release via Cu Nanoparticles Leads to an Increase in Antimicrobial Effects and Hemocompatibility for Short-Term Extracorporeal Circulation. ACS Applied Bio Materials 2019, 2 (6), 2539-2548.
31. Mondal, A.; Douglass, M.; Hopkins, S. P.; Singha, P.; Tran, M.; Handa, H.; Brisbois, E. J., Multifunctional S-Nitroso-N-acetylpenicillamine-Incorporated Medical-Grade Polymer with Selenium Interface for Biomedical Applications. ACS Appl Mater Interfaces 2019, 11 (38), 34652-34662.
32. Pant, J.; Goudie, M. J.; Hopkins, S. P.; Brisbois, E. J.; Handa, H., Tunable Nitric Oxide Release from S-Nitroso-N-acetylpenicillamine via Catalytic Copper Nanoparticles for Biomedical Applications. ACS Appl Mater Interfaces 2017, 9 (18), 15254-15264.
33. Singha, P.; Goudie, M. J.; Liu, Q.; Hopkins, S.; Brown, N.; Schmiedt, C. W.; Locklin, J.; Handa, H., Multipronged Approach to Combat Catheter-Associated Infections and Thrombosis by Combining Nitric Oxide and a Polyzwitterion: a 7 Day In Vivo Study in a Rabbit Model. ACS Appl Mater Interfaces 2020, 12 (8), 9070-9079.
34. Riccio, D. A.; Dobmeier, K. P.; Hetrick, E. M.; Privett, B. J.; Paul, H. S.; Schoenfisch, M. H., Nitric oxide-releasing S-nitrosothiol-modified xerogels. Biomaterials 2009, 30 (27), 4494-502.
35. Singha, P.; Pant, J.; Goudie, M. J.; Workman, C. D.; Handa, H., Enhanced antibacterial efficacy of nitric oxide releasing thermoplastic polyurethanes with antifouling hydrophilic topcoats. Biomater Sci 2017, 5 (7), 1246-1255.
36. Devine, R.; Goudie, M. J.; Singha, P.; Schmiedt, C.; Douglass, M.; Brisbois, E. J.; Handa, H., Mimicking the Endothelium: Dual Action Heparinized Nitric Oxide Releasing Surface. ACS Applied Materials & Interfaces 2020, 12 (18), 20158-20171.
37. Sadrearhami, Z.; Nguyen, T. K.; Namivandi-Zangeneh, R.; Jung, K.; Wong, E. H. H.; Boyer, C., Recent advances in nitric oxide delivery for antimicrobial applications using polymer-based systems. Journal of Materials Chemistry B 2018, 6 (19), 2945-2959.
38. Schairer, D. O.; Chouake, J. S.; Nosanchuk, J. D.; Friedman, A. J., The potential of nitric oxide releasing therapies as antimicrobial agents. Virulence 2012, 3 (3), 271-9.
39. Mesa-Arango, A. C.; Scorzoni, L.; Zaragoza, O., It only takes one to do many jobs: Amphotericin B as antifungal and immunomodulatory drug. Frontiers in Microbiology 2012, 3 (286).
40. Noor, A.; Preuss, C. V., Antifungal Membrane Function Inhibitors (Amphotericin B). In StatPearls, Treasure Island (FL), 2019.
41. Privett, B. J.; Nutz, S. T.; Schoenfisch, M. H., Efficacy of surface-generated nitric oxide against Candida albicans adhesion and biofilm formation. Biofouling 2010, 26 (8), 973-83.
42. Mohamad, A. J.; Zhu, X.; Liu, X.; Pfleging, W.; Torge, M. In Effect of surface topography on hydrophobicity and bacterial adhesion of polystyrene, 2013; IEEE.
43. Yuan, Y.; Hays, M. P.; Hardwidge, P. R.; Kim, J., Surface characteristics influencing bacterial adhesion to polymeric substrates. RSC Advances 2017, 7 (23), 14254-14261.
44. Harter, K.; Levine, M.; Henderson, S. O., Anticoagulation drug therapy: a review. West J Emerg Med 2015, 16 (1), 11-7.
45. Shepherd, G.; Mohorn, P.; Yacoub, K.; May, D. W., Adverse Drug Reaction Deaths Reported in United States Vital Statistics, 1999-2006. Annals of Pharmacotherapy 2012, 46 (2), 169-175.
46. Dhakal, B.; Kreuziger, L. B.; Rein, L.; Kleman, A.; Fraser, R.; Aster, R. H.; Hari, P.; Padmanabhan, A., Disease Burden, Complication Rates, and Health-Care Costs of Heparin-Induced Thrombocytopenia in the USA: A Population-Based Study. The Lancet Haematology 2018, 5(5), e220-e231.
47. Vaughn, M. W.; Kuo, L.; Liao, J. C., Estimation of Nitric Oxide Production and Reactionrates in Tissue by Use of a Mathematical Model. American Journal of Physiology-Heart and Circulatory Physiology 1998, 274 (6), H2163-H2176.
48. Emerson, M.; Momi, S.; Paul, W.; Francesco, P. A.; Page, C.; Gresele, P., Endogenous Nitric Oxide Acts as a Natural Antithrombotic Agent In Vivo by Inhibiting Platelet Aggregation in the Pulmonary Vasculature. Thromb Haemost 1999, 81 (06), 961-966.
49. Singha, P.; Pant, J.; Goudie, M. J.; Workman, C. D.; Handa, H., Enhanced Antibacterial Efficacy of Nitric Oxide Releasing Thermoplastic Polyurethanes with Antifouling Hydrophilic Topcoats. Biomaterials Science 2017, 5 (7), 1246-1255.
50. Weber, M.; Steinle, H.; Golombek, S.; Hann, L.; Schlensak, C.; Wendel, H. P.; Avci-Adali, M., Blood-Contacting Biomaterials: In Vitro Evaluation of the Hemocompatibility. Front Bioeng Biotechnol 2018, 6, 99.
51. Brisbois, E. J.; Major, T. C.; Goudie, M. J.; Meyerhoff, M. E.; Bartlett, R. H.; Handa, H., Attenuation of thrombosis and bacterial infection using dual function nitric oxide releasing central venous catheters in a 9 day rabbit model. Acta Biomaterialia 2016, 44, 304-312.
52. Colletta, A.; Wu, J.; Wo, Y.; Kappler, M.; Chen, H.; Xi, C.; Meyerhoff, M. E., S-Nitroso-N-acetylpenicillamine (SNAP) Impregnated Silicone Foley Catheters: A Potential Biomaterial/Device To Prevent Catheter-Associated Urinary Tract Infections. ACS Biomater Sci Eng 2015, 1 (6), 416-424.
53. Pant, J.; Goudie, M. J.; Chaji, S. M.; Johnson, B. W.; Handa, H., Nitric oxide releasing vascular catheters for eradicating bacterial infection. J Biomed Mater Res B Appl Biomater 2018, 106 (8), 2849-2857.
54. Harmsen, S.; McLaren, A. C.; Pauken, C.; McLemore, R., Amphotericin B is cytotoxic at locally delivered concentrations. Clin Orthop Relat Res 2011, 469 (11), 3016-21.
55. Richards, M. J.; Edwards, J. R.; Culver, D. H.; Gaynes, R. P., Nosocomial infections in medical intensive care units in the United States. National Nosocomial Infections Surveillance System. Crit Care Med 1999, 27 (5), 887-92.
57. Badiee, P.; Hashemizadeh, Z., Opportunistic invasive fungal infections: diagnosis & clinical management. The Indian journal of medical research 2014, 139 (2), 195.

1. An anti-microbial substrate comprising:
a medical grade polymer material forming at least a surface of the substrate, and
an anti-microbial coating comprising a plurality of polyene antimycotic molecules coupled to the medical grade polymer on the substrate surface.

2. The anti-microbial substrate of claim 1, wherein the polyene antimycotic molecules are coupled to the surface of the medical grade polymer via a linker molecule conjugated to the surface of the medical grade polymer, the linker molecule having free primary amines.

3. The anti-microbial substrate of claim 2, wherein the linker has the formula (R¹O)₃Si—R²—NHR³ or (R¹O)₂R¹Si—R²—NHR³, where are R¹ and R² are an alkyl group, and R³ is hydrogen or an alkyl amino group.

4. The anti-microbial substrate of claim 2, wherein the linker molecule is aminopropyltriethoxysilane (APTMS), aminopropyltrimethoxysilane, N-2-(aminoethyl)-3-aminopropyltrimethoxysilane, N-phenyl-3-aminopropyltrimethoxysilane, or N-2-(aminoethyl)-3-aminopropylmethyldimethoxysilane.

5. The anti-microbial substrate of claim 1, wherein the polyene antimycotic molecules comprises amphotericin B (AmB), nystatin, natamycin, or hamycin.

6. The anti-microbial substrate of claim 1, wherein the medical grade polymer comprises a thermoplastic polymer, a thermosetting polymer, silicone, polyvinyl chloride, polyurethane, polyimide, fluoropolymer, rubber, or a thermoplastic elastomer.

7. The anti-microbial substrate of claim 1, wherein the medical grade polymer material is impregnated with a nitric oxide (NO) release agent.

8. The anti-microbial substrate of claim 7, wherein the NO release agent is an S-nitroso thiol of formula O═N—S—R, wherein R is an alkyl moiety or aryl moiety.

9. The anti-microbial substrate of claim 7, wherein the NO release agent is S-nitroso-N-acetylpenicillamine (SNAP), S-nitroso-glutathione, and S-nitroso-N-acetylcysteine.

10. The anti-microbial substrate of claim 7, wherein the NO release agent comprises about 0.1 to about 20% by weight of the medical grade polymer.

11. The anti-microbial substrate of claim 7, wherein the medical grade polymer releases nitric oxide at a rate of from about 0.01×10⁻¹⁰ mol/min-cm² to about 4×10⁻¹⁰ mol/min-cm².

12. The anti-microbial substrate of claim 1, wherein the polyene antimycotic molecules are in an amount of from about 0.5 nmol/cm² to about 5.0 nmol/cm².

13. The anti-microbial substrate of claim 1, wherein the anti-microbial coating comprises a plurality of units having the structure I covalently bonded to the substrate 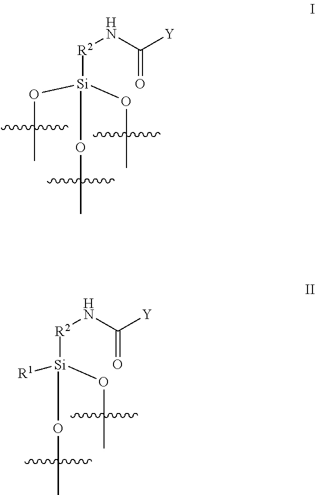
wherein R¹ and R² is an alkyl group, and
Y is a residue of the polyene antimycotic molecule.

14. The anti-microbial substrate of claim 13, wherein R² is C₁ to C₅ alkyl group.

15. The anti-microbial substrate of claim 13, wherein R² is a propyl group and Y is a residue of amphotericin B (AmB).

16. The anti-microbial substrate of claim 13, wherein the medical grade polymer material is impregnated with a nitric oxide (NO) release agent.

17. The anti-microbial substrate of claim 16, wherein the NO release agent is S-nitroso-N-acetylpenicillamine (SNAP), S-nitroso-glutathione, and S-nitroso-N-acetylcysteine.

18. The anti-microbial substrate of claim 13, wherein R² is a propyl group, Y is a residue of amphotericin B (AmB), and the medical grade polymer material is impregnated with S-nitroso-N-acetylpenicillamine (SNAP).

19. A medical device comprising the anti-microbial substrate of claim 1.

20. The medical device of claim 19, wherein the medical device is a catheter or medical tubing comprising an interior lumen having a luminal surface and wherein the luminal surface comprises the anti-microbial coating.

21. The medical device of claim 20, wherein the catheter or medical tubing is selected from the group consisting of: urinary catheters, blood vessel catheters, endotracheal tubing, nephrostomy tubing, colostomy tubing, and medical ports.

22. A method of making an anti-microbial, medical grade polymer substrate, the method comprising:
providing or forming a medical grade polymer substrate having a surface;
treating the surface of the medical grade polymer substrate with O₂ plasma to force —OH groups to the surface;
depositing a plurality of linker molecules with free primary amines on the surface such that the linker molecules conjugate to the —OH groups on the surface;
treating a plurality of polyene antimycotic molecules with 1-ethyl-3-(3-dimethylaminopropyl)carbodiimide (EDC) and N-hydroxysuccinimide (NHS) to convert carboxylic acid groups on the polyene antimycotic molecules to NHS-esters capable of conjugation to primary amines on the linker molecule; and
coupling the plurality of EDC/NHS treated polyene antimycotic molecules to the surface of the medical grade polymer substrate via conjugation of NHS-esters of the polyene antimycotic molecules to primary amines on the linker molecules to form an anti-microbial coating on the surface of the medical grade polymer substrate.

23. The method of claim 22, further comprising impregnating the medical grade polymer substrate with a nitric oxide (NO) release agent.

24. The method of claim 23, wherein impregnating the medical grade polymer substrate with the NO release agent is performed before treating the surface of the medical grade polymer substrate with O₂ plasma.

25. The method of claim 23, wherein the NO release agent is an S-nitroso thiol of formula O═N—S—R, wherein R is an alkyl moiety or aryl moiety.

26. The method of claim 25, wherein the S-nitroso thiol is selected from the group consisting of: S-nitroso-N-acetylpenicillamine (SNAP), S-nitroso-glutathione, and S-nitroso-N-acetylcysteine.

27. A method of treating or preventing a fungal and bacterial infection in a subject associated with a medical device, the method comprising:
administering the medical device of claim 19 to the subject, such that a portion of the device contacts the subject, wherein the anti-microbial coating treats or prevents fungal and bacterial growth on the surface of the substrate and/or on a tissue of the subject adjacent to the device.
